(19) United States
(12) Patent Application Publication
(10) Pub. No.: US 2011/0201858 A1
(43) Pub. Date: Aug. 18, 2011

(54) PROCESS FOR PRODUCING CUMENE

(75) Inventors: Shyh-Yuan Henry Hwang, Needham, MA (US); Dana E. Johnson, Hopkinton, MA (US); Joseph C. Peters, Quincy, MA (US); Chung-Ming Chi, Needham, MA (US); Kevin J. Fallon, Boston, MA (US); Francis A. Demers, Holderness, NH (US)

(73) Assignee: BADGER LICENSING LLC, Cambridge, MA (US)

(21) Appl. No.: 13/119,789

(22) PCT Filed: Sep. 23, 2009
(86) PCT No.: PCT/US2009/057949

§ 371 Date: Mar. 18, 2011

Related U.S. Application Data
(60) Provisional application No. 61/103,066, filed on Oct. 6, 2008.
(60) Provisional application No. 61/107,548, filed on Oct. 22, 2008.

(51) Int. Cl. C07C 2/00

(52) U.S. Cl. 585/314; 585/323

(57) ABSTRACT

In a process for producing cumene from acetone and benzene, a feed stream comprising acetone is contacted with hydrogen in the presence of a hydrogenation catalyst in a first reaction zone under hydrogenation conditions sufficient to convert at least part of the acetone to isopropanol and produce a first liquid effluent stream rich in isopropanol and a first vapor stream rich in unreacted hydrogen. Benzene is then added to at least part of the first liquid effluent stream, without intermediate purification of the first liquid effluent stream, and optionally to at least part of the first vapor stream, to form a second feed stream. The second feed stream is then contacted with an alkylation catalyst in a second reaction zone separate from the first reaction zone under alkylation conditions sufficient to maintain at least part of the second feed stream in the liquid phase and to cause at least part of the isopropanol in the second feed stream to react with the benzene to form cumene and water and produce a second effluent stream comprising at least cumene, water and unreacted benzene. Hydrogen is separated from the first vapor stream and/or the second effluent stream. At least part of the hydrogen is recycled to the first reaction zone and/or purged from the system.

### FIELD

This invention relates to a process for producing cumene and particularly, but not exclusively, to an integrated process for producing cumene and for converting the cumene to phenol.

### BACKGROUND

Cumene is generally produced by the alkylation of benzene with propylene in the presence of an acid catalyst. Early cumene plants used solid phosphoric acid as the catalyst, but more recently most cumene manufacturers have replaced the phosphoric acid with zeolite-based catalysts. Examples of zeolite catalyzed benzene alkylation processes can be found in, for example, U.S. Pat. Nos. 4,185,040; 4,992,606 and 5,073,653.

One problem facing cumene manufacturers is the increasing cost and scarcity of propylene, making it desirable to find alternative C₃ alkylating agents. For example, it is known to produce cumene by alkylating benzene with isopropanol but, with most acid catalysts, the water coproduced in this process negatively impacts the cumene selectivity and the catalyst lifetime.

Most of the cumene produced worldwide is converted to phenol, since phenol has wide application for producing phenolic resins and para, para-bisphenol A; as a selective solvent for refining lubricating oils; and in the manufacture of cyclohexanone, salicylic acid, phenolphthalein, pentachlorophenol, acetophenetidine, picric acid, germicidal paints, and pharmaceuticals; as well as use as a laboratory reagent.

Conversion of cumene to phenol is generally effected by the Hock process, in which the cumene is initially oxidized to form cumene hydroperoxide, which in turn is decomposed or cleaved to produce equimolar amounts of phenol and acetone. The conventional Hock process, which dates back to patents to Allied Chemical and Hercules Chemical Co. in the 1950s, is economical as long as there is adequate demand for the acetone coproduct. However, whereas the demand for phenol remains strong, there has for many years been a surplus of acetone on the world market.

In view of the increasing scarcity of propylene and the over-supply of acetone, there has for some time been interest in developing a process for using the excess acetone as a feedstock for producing cumene.

For example, U.S. Pat. No. 5,015,786 (Araki '786), which is incorporated herein by reference, teaches a process for preparing phenol by the cumene process including the step of converting acetone coproduced with the phenol into isopropanol, thereafter alkylating benzene with the isopropanol and, optionally, with propylene, using a proton-exchanged Y-type zeolite catalyst to produce cumene, thereby forming phenol without the usual acetone coproduct.

Somewhat similar to Araki '786 is U.S. Pat. No. 5,017,729 ('729 Fukuhara), which is also incorporated herein by reference and which teaches a multi-step phenol production process comprising: (a) reacting benzene with propylene in the presence of an aluminum chloride complex to synthesize cumene, (b) oxidizing the cumene of step (a) into cumene hydroperoxide, (c) acid cleaving cumene hydroperoxide into phenol and acetone, (d) hydrogenating the acetone of step (c) into isopropanol, (e) dehydrating the isopropanol of step (d) into propylene, and (f) recycling the propylene of step (e) to step (a). It is also possible to take a propylene product from step (e).

U.S. Pat. No. 5,160,497 (Juguin '497), which is also incorporated herein by reference, teaches still another variation of a phenol production process addressed specifically to dealing with the less-desired acetone coproduct. Thus, the Juguin '497 patent observes (col. 1, lines 58-61) that: “Nowadays, the main handicap of this [cumene-to-phenol] process lies in the obligatory coproduction of 0.61 ton of acetone per ton of phenol, because the demand for phenol increases much more rapidly than that for acetone.” The improvement of the Juguin '497 patent is stated to be (col. 1, line 66-col. 2, line 2) “in partly or totally hydrogenising the acetone produced into isopropyl alcohol, and in recycling at least partly the latter to the stage of alkylation of benzene where, after dehydration into propene, it will be converted again into cumene.”

The Juguin '497 patent further notes, however, that successful practice of this invention is highly catalyst dependent because (col. 2, lines 8-12) the conventional alkylation catalysts “are not adapted to the reaction of alkylation of benzene in the presence of isopropyl alcohol because they are very sensitive to water.” Instead of using conventional aluminum chloride or phosphoric acid catalysts, Juguin '497 turns to a specific class of zeolite catalyst, a dealuminized Y zeolite, which is purported to be stable in the presence of the steam generated by dehydration of isopropyl alcohol.

U.S. Pat. No. 6,512,153 to Cappellazzo et al., which is also incorporated herein by reference, discloses a process for the alkylation of aromatic compounds by reaction with isopropanol, alone or mixed with propylene, wherein the reaction is carried out in the presence of a large pore type zeolite, such as beta, Y, ZSM-12 and mordenite (col. 4, lines 1-3). According to Cappellazzo et al., the concentration of water in the liquid phase of the reactor should never exceed 8,000 ppm w/w.

U.S. Pat. No. 6,841,704 to Sakuth et al., which is also incorporated herein by reference, discloses a process for preparing cumene wherein isopropanol, or a mixture of isopropanol and propylene is reacted with benzene in the presence of a β-zeolite catalyst having a SiO₂/Al₂O₃ molar ratio of greater than 10:1, which process can be integrated in a process for preparing phenol.

U.S. Pat. No. 6,888,035 to Fallon et al., which is also incorporated herein by reference, discloses a method for producing phenol which includes oxidizing cumene to form cumene hydroperoxide and acid cleavage to form cumene, phenol, acetone, and various byproducts, including alpha methylstyrene, followed by a subsequent hydrogenation of at least a part of the acetone and substantially all of the alpha methylstyrene. The resultant isopropanol can be separated by distillation and fed with benzene and propylene to an alkylation reactor for producing cumene.

U.S. Patent Application Publication No. 2005/0075239 to Girotti et al. discloses a process for preparing alkylated aromatic compounds which comprises reacting an aromatic compound with a ketone and hydrogen in the presence of a catalytic composition comprising a solid acid material and copper. The application is mainly focused on the composition of such a dual-function catalyst and does not teach in detail how a reactor system can be operated to achieve high performance. The U.S. Patent Application 2005/0075239 is therefore different from the current invention in which a catalyst composition is used in a reaction zone to hydrogenate acetone to isopropanol, another catalyst composition is used in another reaction zone to react isopropanol and benzene to produce cumene, and the operation of the two reaction zones are taught in details to achieve high performance.

European Patent No. 1,069,099 B1 discloses a process for producing cumene by alkylation of benzene with isopropanol, either alone or in admixture with propylene, in the presence of zeolite beta and under temperature and pressure conditions such that the reaction mixture is completely in the gas phase. The isopropanol is produced by hydrogenation of the acetone coproduced when the cumene is converted to phenol.

However, despite these recent advances, a simple and cost-effective process for converting acetone to cumene, which can readily be incorporated into an integrated cumene and phenol plant, has yet to be developed. The present invention seeks to address this problem.

### SUMMARY

In one aspect, the invention resides in a process for producing cumene from acetone and benzene, the process comprising:

(a) contacting a first feed stream comprising acetone with hydrogen in the presence of a hydrogenation catalyst in a first reaction zone under hydrogenation conditions sufficient to convert at least part of said acetone to isopropanol and produce a first liquid effluent stream rich in isopropanol and a first vapor stream rich in unreacted hydrogen;

(b) adding benzene to at least part of said first liquid effluent stream, without intermediate purification of said first liquid effluent stream, and optionally to at least part of the first vapor stream, to form a second feed stream;

(c) contacting said second feed stream with an alkylation catalyst in a second reaction zone separate from said first reaction zone under alkylation conditions sufficient to maintain at least part of the second feed stream in the liquid phase and to cause at least part of the isopropanol in the second feed stream to react with said benzene to form cumene and water and produce a second effluent stream comprising at least cumene, water and unreacted benzene;

(d) separating hydrogen from said first vapor stream and/or said second effluent stream; and

(e) recycling at least part of the hydrogen separated in (d) to the first reaction zone and/or purging at least part of the hydrogen separated in (d).

Conveniently, said hydrogenation catalyst comprises at least one metal or compound thereof selected from group consisting of copper, nickel, chromium, zinc, platinum, palladium, ruthenium and rhodium.

Conveniently, said conditions in said contacting (a) comprise a temperature of 20° C. to 350° C., a pressure of 100 kPa to 20,000 kPa, and a molar ratio of hydrogen to acetone of 0.1:1 to 100:1.

Conveniently, said alkylation catalyst comprises at least one zeolite catalyst selected from the group comprising ZSM-3, ZSM-4, ZSM-5, ZSM-11, ZSM-12, ZSM-14, ZSM-18, ZSM-20, ZSM-22, ZSM-23, ZSM-35, ZSM-48, zeolite beta, zeolite Y, Ultrastable Y (USY), Dealuminized Y (Deal Y), mordenite, MCM-22, PSH-3, SSZ-25, ERB-1, ITQ-1, ITQ-2, MCM-36, MCM-49, MCM-56, and UZM-8.

In one embodiment, said alkylation catalyst comprises at least one molecular sieve of the MCM-22 family. Conveniently, said molecular sieve of the MCM-22 family has an X-ray diffraction pattern including d-spacing maxima at 12.4±0.25, 6.9±0.15, 3.57±0.07 and 3.42±0.07 Angstroms and typically is selected from MCM-22, PSH-3, SSZ-25, ERB-1, ITQ-1, ITQ-2, MCM-36, MCM-49, MCM-56, UZM-8, and mixtures thereof.

Conveniently, said conditions in said contacting (c) comprise a temperature of 20° C. to 350° C., a pressure of 100 kPa to 20,000 kPa, and a molar ratio of benzene to C₃ alkylating agent (isopropanol plus any added propylene) of 0.1:1 to 100:1.

In one embodiment, the process further comprises recycling at least part of the first liquid effluent stream to the first reaction zone.

In a further embodiment, the process further comprises recycling at least part of said second effluent stream to the second reaction zone.

In yet a further embodiment, the process further comprises:

(i) separating said second effluent stream into a hydrogen-rich second vapor stream, a water-rich aqueous stream and an aromatic stream composed mainly of cumene and unreacted benzene;

(ii) recycling at least part of the aromatic stream to the second reaction zone; and

(iii) recycling at least part of the hydrogen in said hydrogen-rich vapor stream to at least one of said first and second reaction zones and/or purging at least part of the hydrogen in said hydrogen-rich vapor stream.

In one embodiment, said first and second reaction zones are housed within a single reactor.

In a further aspect, the invention resides in an integrated process for producing phenol, the process comprising:

(a) contacting a first feed stream comprising acetone with hydrogen in the presence of a hydrogenation catalyst in a first reaction zone under hydrogenation conditions sufficient to convert at least part of said acetone to isopropanol and produce a first liquid effluent stream rich in isopropanol and a first vapor stream rich in unreacted hydrogen;

(b) adding benzene to at least part of the said first liquid effluent stream, without intermediate purification of said first liquid effluent stream, and optionally to at least part of the first vapor stream, to form a second feed stream;

(c) contacting said second feed stream with an alkylation catalyst in a second reaction zone separate from said first reaction zone under alkylation conditions sufficient to maintain at least part of the second feed stream in the liquid phase and to cause at least part of the isopropanol in the second feed stream to react with said benzene to form cumene and water and produce a second effluent stream comprising at least cumene, water and unreacted benzene;

(d) separating hydrogen from said first vapor stream and/or said second effluent stream;

(e) recycling at least part of said hydrogen from (d) to the first reaction zone and/or purging at least part of the said hydrogen from (d);

(f) separating cumene from said second effluent stream and oxidizing at least part of said cumene to form cumene hydroperoxide;

(g) cleaving at least part of said cumene hydroperoxide to form a cleavage effluent stream containing phenol and acetone; and

(h) separating acetone from said cleavage effluent stream and recycling at least part of acetone to said contacting (a).

In one embodiment, propylene is added to said second feed stream.

### BRIEF DESCRIPTION OF THE DRAWINGS

FIG. 1 is a flow diagram of a process for producing cumene from acetone according to one embodiment of the invention.

FIG. 2 is a flow diagram of a process for producing cumene from acetone according to another embodiment of the invention.

### DETAILED DESCRIPTION OF THE EMBODIMENTS

Described herein is a process for producing cumene from acetone, in which the acetone feed, which is preferably substantially free of benzene, is contacted with hydrogen in the presence of a hydrogenation catalyst in a first reaction zone under hydrogenation conditions sufficient to convert the acetone to isopropanol. The effluent from the first reaction zone comprises a first liquid stream rich in isopropanol and a first vapor stream rich in unreacted hydrogen. At least part of the unreacted hydrogen in the first vapor stream is generally separated from the first vapor stream and then recycled to the first reaction zone and/or purged from the system.

At least part of the isopropanol in the first liquid stream, without intermediate purification but optionally with the addition of propylene, is then contacted with benzene and optionally with part of the first vapor stream, in a second reaction zone separate from, but typically in the same reaction vessel as, the first reaction zone. Contacting in the second reaction zone is conducted in the presence of an alkylation catalyst and under alkylation conditions sufficient to maintain at least part of the second feed stream in the liquid phase and to cause the isopropanol to react with the benzene to form cumene and water. If present, propylene also reacts with the benzene to form cumene.

The effluent from the second reaction zone comprises at least cumene, water, unreacted benzene and optionally unreacted hydrogen from the first vapor stream. If present, the unreacted hydrogen can be removed from second reactor effluent and at least partly recycled to the first reaction zone and/or purged. The remaining liquid effluent from the second reaction zone is separated in to an aqueous phase, which is generally purged, and an aromatic phase containing cumene and unreacted benzene. The cumene is recovered from the aromatic phase and at least part of the remaining unreacted benzene is recycled to the second reaction zone.

Generally, the present process will form part of an integrated scheme for producing phenol, in which case the cumene product is oxidized to cumene hydroperoxide, the hydroperoxide is cleaved to produce phenol and acetone and the acetone is recycled to the first reaction zone.

### Acetone Hydrogenation

The acetone hydrogenation step of the present process is effected by contacting an acetone-containing feed, such as an acetone-containing stream from a co-located phenol plant, purified acetone produced from a co-located phenol plant, or acetone acquired from other source, with a hydrogenation catalyst. Generally the catalyst is Raney nickel, but other useful catalysts include nickel, copper-chromium, Raney nickel-copper, copper-zinc and platinum group metals, for example, platinum, palladium, ruthenium, rhodium, and similar metals on active carbon, aluminum and other carriers. The reaction temperature may range from 20° C. to 350° C., but more generally is between 40° C. and 250° C., such as between 60° C. and 200° C. The hydrogenation may be carried out by either liquid, gas, or mixed gas-liquid phase reaction. The pressure may range from 100 kPa to 20,000 kPa, such as from 500 to 10,000 kPa. The hydrogen gas is generally present in a molar ratio relative to the acetone reactant of from 0.1:1 to 100:1, such as from 1:1 to 10:1.

The hydrogenation may be carried out in the presence or absence of a reaction medium. Examples of suitable solvents include alcohols such as methanol, ethanol, propanol, and butanol. Also useful is isopropanol which is a hydrogenation product of acetone. A Iso useful are glycols such as ethylene glycol, propylene glycol, diethylene glycol, and triethylene glycol; and ethers such as diisopropyl ether, dibutyl ether, ethylene glycol dimethyl ether, diglyme (diethylene glycol dimethyl ether) and triglyme. Aprotic polar solvents may also be used, for example, dimethylformamide, dimethylacetamide, acetonitrile, and dimethyl sulfoxide. Also useful are saturated hydrocarbons such as hexane, heptane, cyclopentane, and cyclohexane. Water can also be used as a solvent in the hydrogenation reaction.

The hydrogenation step may be carried out batchwise or on a continuous basis. Depending on the shape of a particular catalyst used, the reaction may be carried out in a fluidized bed using powder catalyst or a fixed bed using granular catalyst. Fixed bed operation is preferred in view of ease of separation of the catalyst from the reaction mixture and simplicity of the reaction system.

The hydrogenation reaction is exothermic and, to avoid excessive temperature rise, part of the liquid phase reaction effluent, composed mainly of isopropanol, can be cooled and recycled to hydrogenation reactor inlet. In one embodiment, the weight ratio of liquid recycle to acetone feed is between 1:1 and 100:1.

The hydrogenation reaction effluent also includes a vapor phase component rich in unreacted hydrogen but also generally containing other light by-products. This vapor phase component is separated from the liquid phase effluent and, depending on the amount of unreacted hydrogen therein, the vapor phase component can be recycled to the hydrogenation reactor inlet so as to reduce the level of hydrogen in the isopropanol-containing feed to the alkylation step and/or partially or completely purged.

Alkylation of Benzene with Isopropanol

The alkylation step of the present process is effected by contacting the isopropanol-containing effluent from the hydrogenation step, optionally together with added propylene, with benzene in the presence of an alkylation catalyst in a second reaction zone of the reactor, separate from the first reaction zone of the reactor. Typically the molar ratio of benzene added to the second reaction zone to the C₃ alkylating agent (isopropanol plus any added propylene) added to the second reaction zone is maintained within the range of 0.1:1 to 100:1, such as 1:1 to 10:1.

The alkylation reaction is conducted at a temperature of 20° C. to 350° C., for example 60° C. to 300° C., and a pressure of 100 kPa to 20,000 kPa, for example 500 kPa to 10,000 kPa, so that at least part of the reaction mixture is maintained in the liquid phase during the process. In addition, the alkylation can be conducted in the presence or absence of hydrogen. Hydrogen, if present, can be either added directly to the alkylation reaction zone or present in the hydrogenation reaction zone effluent. It is found that hydrogen assists in removing the water coproduced with cumene in the alkylation step from the liquid phase reaction medium thereby reducing the contact between the catalyst and the water and hence any tendency for the water to deactivate the catalyst. A similar effect can also be achieved by adjusting the temperature and/or pressure of the alkylation zone to cause more water to be removed from liquid phase to vapor phase or by increasing the recycle of the alkylation effluent, after said effluent stream is cooled and water is removed, to the alkylation step thus reducing the concentration of water in the alkylation zone. For some catalysts, the presence of hydrogen in said alkylation step also reduces the deactivation caused by coke formation on the catalyst. Excessive hydrogen should, however, be avoided since it can lead to undesirable loss of benzene to cyclohexane.

In an alternative embodiment of the present process, substantially all of the hydrogen in the first effluent stream exiting the hydrogenation zone is removed prior to the first effluent stream entering the alkylation catalyst bed.

The catalyst employed in the alkylation step may comprise at least one medium pore molecular sieve having a Constraint Index of 2-12 (as defined in U.S. Pat. No. 4,016,218). Suitable medium pore molecular sieves include ZSM-5, ZSM-11, ZSM-12, ZSM-22, ZSM-23, ZSM-35, and ZSM-48. ZSM-5 is described in detail in U.S. Pat. No. 3,702,886 and Re. 29,948. ZSM-11 is described in detail in U.S. Pat. No. 3,709,979. ZSM-12 is described in U.S. Pat. No. 3,832,449. ZSM-22 is described in U.S. Pat. No. 4,556,477. ZSM-23 is described in U.S. Pat. No. 4,076,842. ZSM-35 is described in U.S. Pat. No. 4,016,245. ZSM-48 is more particularly described in U.S. Pat. No. 4,234,231.

Alternatively, the alkylation catalyst may comprise one or more large pore molecular sieves having a Constraint Index less than 2. Suitable large pore molecular sieves include zeolite beta, zeolite Y, Ultrastable Y (USY), Dealuminized Y (Deal Y), mordenite, ZSM-3, ZSM-4, ZSM-18, and ZSM-20. Zeolite ZSM-14 is described in U.S. Pat. No. 3,923,636. Zeolite ZSM-20 is described in U.S. Pat. No. 3,972,983. Zeolite Beta is described in U.S. Pat. No. 3,308,069, and Re. No. 28,341. Low sodium Ultrastable Y molecular sieve (USY) is described in U.S. Pat. Nos. 3,293,192 and 3,449,070. Dealuminized Y zeolite (Deal Y) may be prepared by the method found in U.S. Pat. No. 3,442,795. Zeolite UHP-Y is described in U.S. Pat. No. 4,401,556. Mordenite is a naturally occurring material but is also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104.

Preferably, however, the alkylation catalyst comprises at least one molecular sieve of the MCM-22 family. As used herein, the term “molecular sieve of the MCM-22 family” (or “material of the MCM-22 family” or “MCM-22 family material” or “MCM-22 family zeolite”) includes one or more of
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Molecular sieves of the MCM-22 family include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Materials of the MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), and mixtures thereof.

The above molecular sieves may be used as the alkylation catalyst without any binder or matrix, i.e., in so-called self-bound form. Alternatively, the molecular sieve may be composited with another material which is resistant to the temperatures and other conditions employed in the alkylation reaction. Such materials include active and inactive materials and synthetic or naturally occurring zeolites as well as inorganic materials such as clays and/or oxides such as alumina, silica, silica-alumina, zirconia, titania, magnesia or mixtures of these and other oxides. The latter may be either naturally occurring or in the form of gelatinous precipitates or gels including mixtures of silica and metal oxides. Clays may also be included with the oxide type binders to modify the mechanical properties of the catalyst or to assist in its manufacture. Use of a material in conjunction with the molecular sieve, i.e., combined therewith or present during its synthesis, which itself is catalytically active may change the conversion and/or selectivity of the catalyst. Inactive materials suitably serve as diluents to control the amount of conversion so that products may be obtained economically and orderly without employing other means for controlling the rate of reaction. These materials may be incorporated into naturally occurring clays, e.g., bentonite and kaolin, to improve the crush strength of the catalyst under commercial operating conditions and function as binders or matrices for the catalyst. The relative proportions of molecular sieve and inorganic oxide matrix vary widely, with the sieve content ranging from about 1 to about 90 percent by weight and more usually, particularly, when the composite is prepared in the form of beads, in the range of about 2 to about 80 weight percent of the composite.

The alkylation step may be carried out batchwise or on a continuous basis. Moreover, the reaction may be carried out in a fixed or moving bed. Fixed bed operation is, however, preferred, typically with the alkylation reaction zone comprising one or a plurality of series-connected beds of alkylation catalysts. Moreover, although the alkylation reaction zone is separate from the hydrogenation zone, both zones are contained in a single reaction vessel.

The alkylation step is generally operated so as to achieve substantially complete conversion of the C₃ alkylating agent (isopropanol plus any added propylene) and hence the effluent from the second reaction zone is composed mainly of cumene, coproduced water, unreacted benzene, and other reaction products. Hydrogen will be present in the effluent if it is present in the feed. Part of the effluent is typically recycled to the alkylation zone in order to control the reaction temperature. It is, however, important to avoid build-up of water in the alkylation reactor and hence the alkylation effluent is dewatered before the effluent is recycled. If hydrogen is present in the effluent, this is typically achieved by passing the effluent into a vapor/liquid separator to divide the effluent into a hydrogen-rich vapor stream and a hydrogen-depleted liquid stream. The hydrogen-rich vapor stream can then be purged. Alternatively, the hydrogen-rich vapor stream can be recycled to at least one of said first and second reaction zones, generally after being compressed and cooled to separate any entrained water and aromatics. The hydrogen-depleted liquid stream is separated into a water-rich aqueous stream and a water-depleted aromatic stream comprising cumene, unreacted benzene, and other reaction products. Part of the aromatic stream is recycled to the alkylation reaction zone. If hydrogen is not present in the effluent, the effluent stream can be cooled, separated into a water-rich aqueous stream and a water-depleted aromatic stream comprising cumene, unreacted benzene, and other reaction products. Part of the aromatic stream is recycled to the alkylation reaction zone.

### Integrated Process for Producing Phenol

In one embodiment, the present process for converting acetone to cumene forms part of an integrated process for producing phenol. In such an integrated process, the cumene produced in the alkylation step of the present process is oxidized to form cumene hydroperoxide and the cumene hydroperoxide is cleaved to form a cleavage effluent stream containing phenol and acetone. An acetone-containing stream is then separated from the cleavage effluent stream and recycled back to the hydrogenation step of the present process. Details of the cumene oxidation and cleavage steps can be found in, for example, U.S. Pat. No. 5,017,729, which is incorporated herein by reference.

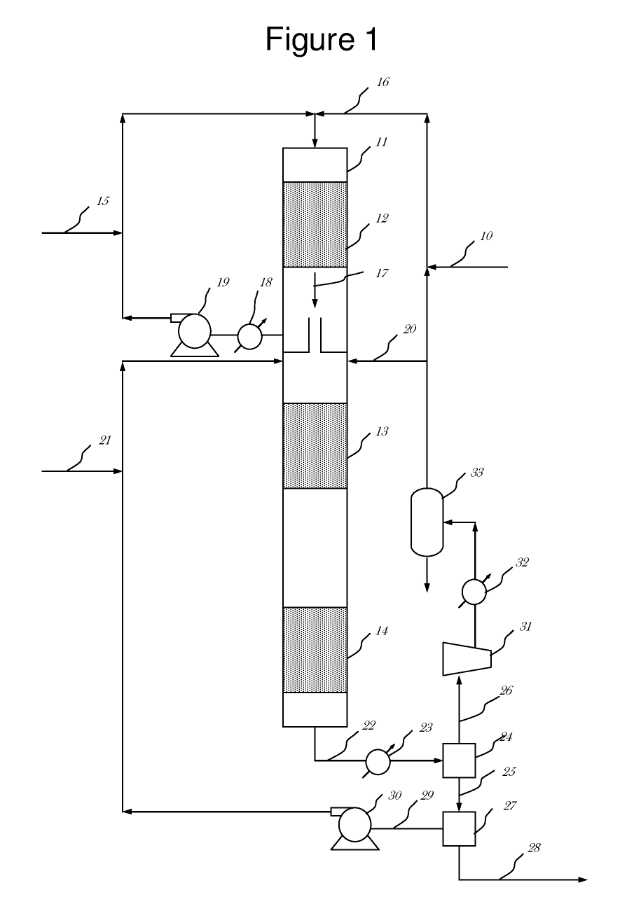

Referring now to the drawings, one embodiment of the present process for converting acetone to cumene is illustrated in FIG. 1, in which a single reactor 11 houses a single fixed bed 12 of hydrogenation catalyst and two fixed beds 13, 14 of alkylation catalyst connected in series. An acetone feed 15 and a hydrogen feed 10, 16 are supplied to the inlet end of the hydrogenation catalyst bed 12 and an isopropanol-containing effluent stream 17 is withdrawn from the opposite end of the bed 12. Part of the liquid in effluent stream 17 is cooled by passage through a heat exchanger 18 and is then recycled by a pump 19 back to the inlet of the hydrogenation catalyst bed 12.

The remainder of the effluent stream 17 is combined with a benzene feed 21 and a hydrogen feed 20 and is fed sequentially to the alkylation catalyst beds 13, 14 where the isopropanol reacts with the benzene to produce cumene and water. The effluent 22 from the downstream alkylation catalyst bed 14 is cooled by passage through a heat exchanger 23 and is then passed to a vapor/liquid separator 24. The separator 24 divides the effluent 22 into a liquid stream 25 composed mainly of cumene, unreacted benzene and water and a vapor stream 26 composed mainly of hydrogen. The liquid stream 25 is passed to a decanter 27 where it is separated into a water-rich aqueous phase 28 and an aromatic phase 29 composed mainly of cumene and unreacted benzene. The water-rich aqueous phase and part of the aromatic phase is discharged (from the bottom of the decanter as the net reactor effluent (28) while part of the aromatic phase (29) is recycled by a pump 30 to the alkylation stage. Cumene is recovered from the aromatic phase 27 generally by distillation (not shown).

The vapor stream 26 composed mainly of hydrogen is fed to a compressor 31 and is then cooled by passage through a heat exchanger 32. Compressing and cooling the vapor steam 26 causes the water and any entrained hydrocarbons to condense so that they can be removed in a knock-out drum 33 before the hydrogen is recycled back to the alkylation and/or hydrogenation step.

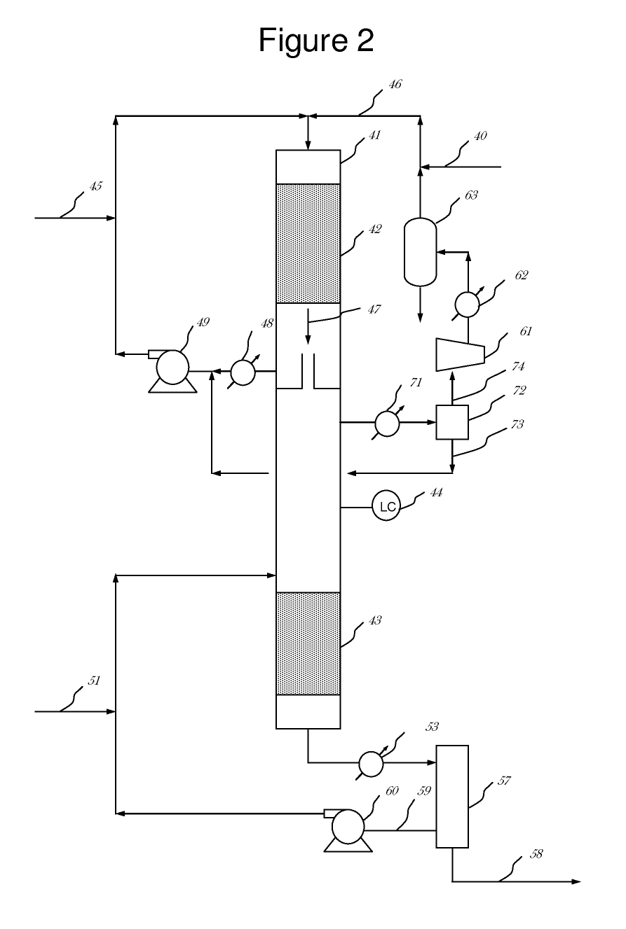

Another embodiment of the present process for converting acetone to cumene is illustrated in FIG. 2, in which a single reactor 41 houses a single fixed bed 42 of hydrogenation catalyst and one fixed bed 43 of alkylation catalyst. An acetone feed 45 and a hydrogen feed 40, 46 are supplied to the inlet end of the hydrogenation catalyst bed 42 and an isopropanol-containing effluent stream 47 is withdrawn from the opposite end of the bed 42. Part of the liquid in effluent stream 47 is cooled by passage through a heat exchanger 48 and is then recycled by a pump 49 back to the inlet of the hydrogenation catalyst bed 42.

The gaseous part of effluent stream 47, containing hydrogen, is cooled by passage through a heat exchanger 71 and is then fed to a vapor/liquid separator 72 where it is separated into a liquid stream 73 and a vapor stream 74. The liquid stream 73 is sent to the inlet of pump 49 and is recycled back to the inlet of the hydrogenation catalyst bed 42. The vapor stream 74 is sent to compressor 61 and is then cooled by passage through a heat exchanger 62. Compressing and cooling the vapor steam 74 causes the water and any entrained hydrocarbons to condense so that they can be removed in a knock-out drum 63 before the hydrogen is recycled back to the hydrogenation step.

The remainder of the effluent stream 47, which is substantially free of gaseous hydrogen, is combined with a benzene feed 51 and is fed to alkylation catalyst bed 43 where the isopropanol reacts with the benzene to produce cumene and water. A level controller 44 is employed to maintain a liquid level above the alkylation catalyst bed 43 so the alkylation catalyst is in essentially complete liquid phase. The effluent from the alkylation catalyst bed 43 is cooled by passage through a heat exchanger 53 and is then passed to a decanter 57 where it is separated into a water-rich aqueous phase and an aromatic phase composed mainly of cumene and unreacted benzene. The water-rich aqueous phase and part of the aromatic phase is discharged from the bottom of the decanter as the net reactor effluent (58) while part of the aromatic phase (59) is recycled by a pump 60 to the alkylation stage.

The invention, in the embodiment depicted in FIG. 1, will now be more particularly described with reference to the following Examples.

### Example 1

A Raney Nickel catalyst 3110 provided by Grace Davison was tested as the hydrogenation catalyst. An acetone feed containing 35 wt % acetone and 65 wt % isopropanol was used. The hydrogenation reaction was carried out at (Acetone+Isopropanol) WHSV of 0.6 hr⁻¹, hydrogen to (Acetone+Isopropanol) feed molar ratio of 6:1 molar, a inlet temperature of 108° C., an effluent circulation to feed ratio of 11:1, and a reactor pressure of about 3,600 kPa. Two alkylation zones were used, each containing an MCM-22 catalyst provided by ExxonMobil. The alkylation reaction was carried out at (Acetone+Isopropanol) WHSV of 0.6 h⁻¹, benzene to (Acetone+Isopropanol) feed molar ratio of 3:1, reactor circulation to feed ratio of 2:1, reactor outlet pressure of 3,600 kPa, and inlet temperatures=142° C. and 195° C. Both the hydrogenation and alkylation zones mentioned above were operated in partial liquid phase and housed in the same reactor. Acetone and isopropanol conversions were both 100%. Aromatics selectivity was 99%.

### Examples 2 to 6

The process of Example 1 was repeated with a pure acetone feed and with the conditions in the hydrogenation and alkylation steps being varied as shown in Table 1. The results are also shown in Table 1.

TABLE 1

| | Example | | | | |
|---|---|---|---|---|---|
| | 2 | 3 | 4 | 5 | 6 |
| Hydrogenation Catalyst | Raney 3110 | | | | |
| Acetone WHSV (1/hr) relative to | 0.6 | | | | |
| hydrogenation catalyst | | | | | |
| Feed hydrogen/acetone feed ratio, | 6:1 | 3:1 | 4:1 | 2:1 | 2:1 |
| molar (including fresh and recycle | | | | | |
| hydrogen) | | | | | |
| Liquid recycle/acetone feed ratio, | 14:1 | 22:1 | 13:1 | 27:1 | 27:1 |
| wt/wt | | | | | |
| Hydrogenation bed inlet | 102 | 120 | 120 | 115 | 115 |
| temperature, ° C. | | | | | |
| Acetone conversion, % | 99 | 100 | 99 | 100 | 100 |
| Alkylation catalyst | MCM-22 | | | | |
| IPA WHSV (1/hr) relative to | 0.6 | | | | |
| alkylation catalyst | | | | | |
| Benzene/IPA feed ratio, molar | 3:1 | 3:1 | 3:1 | 3:1 | 2:1 |
| Total hydrogen/IPA feed ratio, | 5:1 | 5:1 | 5:1 | 5:1 | 4:1 |
| molar (including H₂ from | | | | | |
| hydrogenation reactor) | | | | | |
| Liquid recycle/(benzene + IPA) feed | 2:1 | 4:1 | 4:1 | 5:1 | 7:1 |
| ratio, wt/wt | | | | | |
| Alkylation bed inlet temperatures, | 140 | 160 | 159 | 170 | 170 |
| ° C. | 195 | 205 | 205 | 215 | 215 |
| Reactor outlet pressure, kPa | 3,617 | 4,583 | 4,583 | 4,583 | 4,583 |
| IPA Conversion, % | 99 | 99 | 99 | 100 | 100 |
| Cumene Selectivity, % | 71 | 70 | 70 | 68 | 60 |
| Aromatics selectivity, % | 99 | 99 | 99 | 99 | 99 |

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for producing cumene from acetone and benzene, the process comprising:
(a) contacting a first feed stream comprising acetone with hydrogen in the presence of a hydrogenation catalyst in a first reaction zone under hydrogenation conditions sufficient to convert at least part of said acetone to isopropanol and produce a first liquid effluent stream rich in isopropanol and a first vapor stream rich in unreacted hydrogen;
(b) adding benzene to at least part of said first liquid effluent stream, without intermediate purification of said first liquid effluent stream, and optionally to at least part of the first vapor stream, to form a second feed stream;
(c) contacting said second feed stream with an alkylation catalyst in a second reaction zone separate from said first reaction zone under alkylation conditions sufficient to maintain at least part of the second feed stream in the liquid phase and to cause at least part of the isopropanol in the second feed stream to react with said benzene to form cumene and water and produce a second effluent stream comprising at least cumene, water and unreacted benzene;
(d) separating hydrogen from said first vapor stream and/or said second effluent stream; and
(e) recycling at least part of the hydrogen separated in (d) to the first reaction zone and/or purging at least part of the hydrogen separated in (d).

2. The process of claim 1 wherein said hydrogenation catalyst comprises at least one metal or compound thereof selected from group consisting of copper, nickel, chromium, zinc, platinum, palladium, ruthenium and rhodium.

3. The process of claim 1 or claim 2 wherein said conditions in said contacting (a) comprise a temperature of 20° C. to 350° C., a pressure of 100 kPa to 20,000 kPa, and a molar ratio of hydrogen to acetone of 0.1:1 to 100:1.

4. The process of any preceding claim wherein said conditions in said contacting (c) comprise a temperature of 20° C. to 350° C., a pressure of 100 kPa to 20,000 kPa, and a molar ratio of benzene to C₃ alkylating agent (isopropanol plus any added propylene) fed to said second reaction zone of 0.1:1 to 100:1.

5. The process of any preceding claim wherein said alkylation catalyst comprises at least one zeolite catalyst selected from the group consisting of ZSM-3, ZSM-4, ZSM-5, ZSM-11, ZSM-12, ZSM-14, ZSM-18, ZSM-20, ZSM-22, ZSM-23, ZSM-35, ZSM-48, zeolite beta, zeolite Y, Ultrastable Y (USY), Dealuminized Y (Deal Y), mordenite, MCM-22, PSH-3, SSZ-25, ERB-1, ITQ-1, ITQ-2, MCM-36, MCM-49, MCM-56, and UZM-8.

6. The process of any preceding claim wherein said alkylation catalyst comprises a molecular sieve of the MCM-22 family.

7. The process of any preceding claim and further comprising recycling at least part of the first liquid effluent stream to the first reaction zone.

8. The process of any preceding claim and further comprising:
(i) separating said first vapor stream rich into a hydrogen-rich gaseous stream and a hydrocarbon-containing liquid stream; and
(ii) recycling at least part of the hydrogen in said hydrogen-rich gaseous stream to said first reaction zone and/or purging at least part of the hydrogen in said hydrogen-rich gaseous stream.

9. The process of claim 8 and further comprising recycling at least part of said hydrocarbon-containing liquid stream to the first reaction zone.

10. The process of any preceding claim and further comprising recycling at least part of said second effluent stream to the second reaction zone.

11. The process of any preceding claim and further comprising:
(i) separating said second effluent stream into a water-rich aqueous stream and an aromatic stream composed mainly of cumene and unreacted benzene; and
(ii) recycling at least part of the aromatic stream to the second reaction zone.

12. The process of any preceding claim and further comprising:
(i) separating said second effluent stream into a hydrogen-rich vapor stream and a liquid stream; and
(ii) recycling at least part of said liquid stream to said second reaction zone.

13. The process of any preceding claim and further comprising:
(i) separating said second effluent stream into a hydrogen-rich vapor stream, a water-rich aqueous stream, and an aromatic stream composed mainly of cumene and unreacted benzene; and
(ii) recycling at least part of the hydrogen in said hydrogen-rich vapor stream to at least one of said first and second reaction zones and/or purging at least part of the hydrogen in said hydrogen-rich vapor stream.

14. The process of any preceding claim and further comprising:
(i) separating said second effluent stream into a hydrogen-rich vapor stream, a water-rich aqueous stream, and an aromatic stream composed mainly of cumene and unreacted benzene;
(ii) recycling at least part of the aromatic stream to the second reaction zone; and
(iii) recycling at least part of the hydrogen in said hydrogen-rich vapor stream to at least one of said first and second reaction zones and/or purging at least part of the hydrogen in said hydrogen-rich vapor stream.

15. The process of any preceding claim and further comprising:
(f) separating cumene from said second effluent stream and oxidizing at least part of said cumene to form cumene hydroperoxide;
(g) cleaving at least part of said cumene hydroperoxide to form a cleavage effluent stream containing phenol and acetone; and
(h) separating acetone from said cleavage effluent stream and recycling at least part of acetone to said contacting (a).

16. The process of any preceding claim wherein propylene is added to said second feed stream.

17. The process of any preceding claim wherein said first and second reaction zones are housed within a single reactor.
